(19) United States
(12) Patent Application Publication
(10) Pub. No.: US 2020/0381116 A1
(43) Pub. Date: Dec. 3, 2020

(54) SYSTEM AND METHOD FOR PROVIDING PERSONAL HEALTH ADVICE BASED ON MULTIMODAL ORAL SENSING DATA

(71) Applicant: Lalitha Ratna Pingali, Bangalore (IN)

(72) Inventor: Lalitha Ratna Pingali, Bangalore (IN)

(21) Appl. No.: 16/835,855

(22) Filed: Mar. 31, 2020

(30) Foreign Application Priority Data
Jun. 3, 2019 (IN) 201941021967

(51) Int. Cl. G16H 50/20; A61B 5/00; G16H 50/50

(52) U.S. Cl. CPC G16H 50/20; A61B 5/0088; A61B 5/682; A61B 5/0062; A61B 2562/0219; A61B 5/0022; A61B 5/7435; G16H 50/50; A61B 2562/0271; A61B 5/7264

(57) ABSTRACT

The present invention provides a system and method for providing personal health advice based on multimodal oral sensing data obtained from a plurality of user(s). The system comprises a handheld multimodal oral scanning device (101) for scanning and collecting oral health data which is transmitted to a user interface device (102) through a wireless network infrastructure. Further, an oral scanning and feedback module (103) provides real-time guidance and feedback to user(s) regarding the optimum usage of the multimodal oral scanning device (101). The collected oral health data is stored in a dynamic oral data aggregation module (104) and pre-processed by a disease classifier module (105) to detect the presence or absence of one or more disease(s) after which a personal health advice generation module (106) provides a personalized primary level advice to the user(s).

### CROSS-REFERENCE TO RELATED APPLICATIONS

The present application claims priority to Indian Application No: 201941021967, filed Jun. 3, 2019, herein incorporated by reference in its entirety.

### PREAMBLE TO THE DESCRIPTION

The following specification particularly describes the invention and the manner in which it is to be performed:

### DESCRIPTION OF THE INVENTION

### Technical field of the Invention

The present invention discloses a system and method for providing personal health advice based on multimodal oral sensing data obtained from a plurality of user(s). Further, the present invention enables the application of machine learning and/ or pattern recognition algorithm(s) to the oral health data obtained from a plurality of user(s) and detects the presence or absence of one or more disease(s).

### Background of the Invention

Doctors believe that examining the mouth of an individual may indicate the health of different organs and provide an early warning to the onset of several systemic diseases. Specialized oral scanners which are available in dental clinics are largely employed for capturing the three-dimensional view of the user(s)' mouth and do not indicate further utility. Moreover, commercially available oral scanners may not be suitable for user(s)' personal utility due to their complexity and unaffordability.

A vast majority of the population in developing countries such as India live in rural villages which have a few low-scale primary healthcare establishments which are sporadically visited by dentist(s). Over 70% of India's population reside in rural areas wherein the dentist to population ratio is 1:150000. A majority of this population are seldom blessed with an opportunity of consulting a medical practitioner(s) due to which the oral health data corresponding to the lower privileged population is not accessible to medical researchers.

The Patent Application number U.S. Pat. No. 8,989,567B1 titled “Dental scanner device and system and methods of use” discloses a 3D scanning device and system especially useful in the field of stomatology, dentistry, or orthodontics, and particularly to dental prosthetics manufacturing. The device and system of the invention is particularly applicable for imaging the surface characteristics of an object, including arbitrarily shaped objects, such as dental structures (e.g., teeth, gingiva, and the like), for generating a three-dimensional (3D) image and surface model of the object or objects. More specifically, the subject invention includes an intraoral 3D dental scanning device and method for imaging and visualizing teeth or gingivae surfaces, including the conformation thereof, useful for generating dental models and the manufacture of dental prosthetics therefrom.

The Patent Application number CN106295139A titled “Tongue body self-diagnosis health cloud service system based on deep convolution neural network” discloses a tongue body self-diagnosis health cloud service system based on a deep convolution neural network. The system mainly comprises a convolution neural network used for deep learning and training recognition, a tongue body segmentation method based on the deep convolution neural network, the deep convolution neural network used for tongue image classification and a health cloud service platform used for conducting self-diagnosis according to a recognized tongue image type. According to the tongue body self-diagnosis health cloud service system based on the deep convolution neural network, the automation and intelligent level of watching the tongue for diagnosis on the basis of a mobile internet can be effectively improved, more people can understand and participate in self-service health detection, evaluation and guidance, the health consciousness of the public is improved, and the self- health management ability is increased.

Hence, there exists a need for a simple and affordable system for obtaining the oral health data of a plurality of user(s) and providing a primary level personalized health advice to the user(s).

### Summary of the Invention

The present invention overcomes the drawbacks of the prior art by disclosing a system and method for providing personal health advice based on multimodal oral sensing data obtained from a plurality of user(s). The system comprises a handheld multimodal oral scanning device with a plurality of sensors for scanning and collecting a plurality of oral health data. The multimodal oral scanning device is interfaced with a user interface device, wherein the oral health data collected by the multimodal oral scanning device is transmitted to the user interface device through a wireless network infrastructure. Further, an oral scanning and feedback module is interfaced with the user interface device for providing real-time guidance and feedback to a user(s) regarding the optimum usage of the multimodal oral scanning device.

The oral health data which is obtained from a plurality of user(s) through the oral scanning and feedback module, is stored in a dynamic oral data aggregation module which is a central database that may be accessed by a pre-defined number of designated authority(ies). The oral health data which is retrieved from the dynamic oral data aggregation module is pre-processed by a disease classifier module after which at least one machine learning and/or pattern recognition algorithm is implemented to detect the presence or absence of one or more disease(s). The results obtained at the disease classifier module are analyzed by a personal health advice generation module which provides a primary level advice to the user(s) depending on the results obtained in the disease classifier module.

The present invention also discloses a method for providing personal health advice based on multimodal oral sensing data, wherein the method comprises the steps of obtaining oral health data of a user(s) through the multimodal oral scanning device. The method allows real-time guidance and feedback to be provided to the user(s) through the oral scanning and feedback module present in the user interface device. Further, the data pertaining to the user(s)' health is obtained through a pre-defined set of questions provided by the oral scanning and feedback module after which the oral health data is stored in the dynamic oral data aggregation module. The method enables the oral health data to be retrieved from the dynamic oral data aggregation module and pre-processed at the disease classifier module, wherein at least one machine learning and/or pattern recognition algorithm(s) is applied for detecting the presence or absence of one or more disease(s). Based on the results obtained at the disease classifier module, a primary level personalized health advice is provided by the personal health advice generation module.

The present invention provides a system and method which is employed to detect a plurality of health problems based on the oral health data obtained from a plurality of user(s) and provide a personalized primary level advice to the user(s) regarding their overall health. This portable, user-friendly and cost-effective solution enables doctors to monitor their patient(s)' health from a remote location. Further, the system is modularly designed to accommodate the latest technology and infrastructure for optimum usage.

### BRIEF DESCRIPTION OF THE DRAWINGS

The foregoing and other features of embodiments will become more apparent from the following detailed description of embodiments when read in conjunction with the accompanying drawings. In the drawings, like reference numerals refer to like elements.

FIG. 1 illustrates a block diagram of a system employed to provide personal health advice based on multimodal oral sensing data obtained from a plurality of user(s).

FIG. 2 illustrates a method for providing personal health advice for a plurality of user(s) based on the oral health data which is obtained using a multimodal oral scanning device.

FIG. 3 illustrates side view of the multimodal oral scanning device which is employed for scanning and collecting oral health data for a plurality of user(s).

### DETAILED DESCRIPTION OF THE INVENTION

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

The present invention provides a system and method for providing personal health advice based on multimodal oral sensing data obtained from a plurality of user(s). The system comprises a handheld multimodal oral scanning device for scanning and collecting a plurality of oral health data which is transmitted to a user interface device through a wireless network infrastructure. Further, an oral scanning and feedback module provides real-time guidance and feedback to a user(s) regarding the optimum usage of the multimodal oral scanning device. The collected oral health data is stored in a dynamic oral data aggregation module and pre-processed by a disease classifier module to detect the presence or absence of one or more disease(s). The results obtained at the disease classifier module are analyzed by a personal health advice generation module which provides a personalized primary level advice to the user(s).

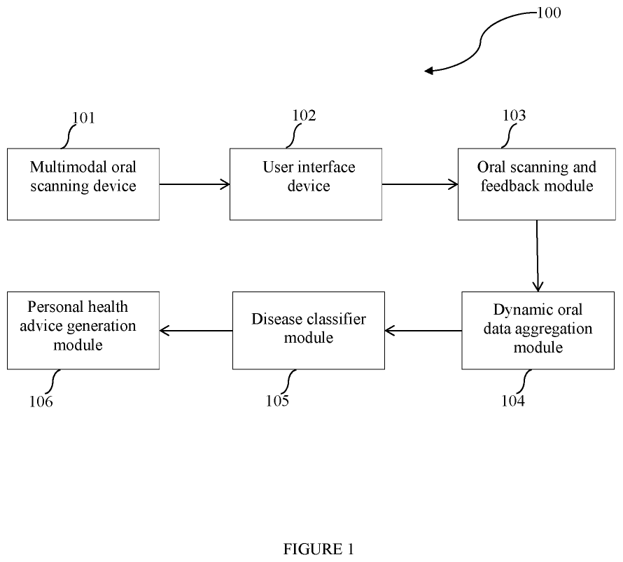

FIG. 1 illustrates a block diagram of a system employed to provide personal health advice based on multimodal oral sensing data obtained from a plurality of user(s). The system (100) comprises a multimodal oral scanning device (101) for scanning and collecting a plurality of oral health data, wherein the multimodal oral scanning device (101) is a handheld device comprising a plurality of sensors such as camera, breath sensor, temperature sensor, at least one accelerometer(s) and so on. The multimodal oral scanning device (101) comprises at least one programmable microcontroller(s) which captures, synchronizes, processes, integrates and transmits the oral health data captured by the multimodal oral scanning device (101) to a user interface device (102) such as a mobile phone through a wireless network infrastructure.

According to one or more embodiments of the present invention, the user interface device (102) is interfaced with an oral scanning and feedback module (103) for providing real-time guidance and feedback to a user(s) regarding the optimum usage of the multimodal oral scanning device (101). The real-time guidance and feedback provided by the oral scanning and feedback module (103) may be in the form of text or a plurality of multi-media formats. The oral scanning and feedback module (103) enables the user(s) to perform a thorough scan of their mouth which covers different regions such as the teeth, gums, tongue, the roof of the mouth, the inside surfaces of the cheeks and so on. The oral scanning and feedback module (103) may consider the previous scans which were executed by the user(s) and guide them to perform an effective scan to yield accurate results. Further, the oral scanning and feedback module (103) comprises a feedback mechanism which provides real-time guidance to the user(s) while they're performing a scan. Further, the oral scanning and feedback module (103) may collect responses from the user(s) regarding their overall health and well-being though a questionnaire which may seek information such as user(s)'present health, pain if any, previous meal, brushing pattern, flossing and so on. The oral health data which is collected by the oral scanning and feedback module (103) is transmitted to a dynamic oral data aggregation module (104) over a secure cloud-based infrastructure.

According to one or more embodiments of the present invention, the dynamic oral data aggregation module (104) is a central database which receives and stores oral health data of a plurality of user(s) that is transmitted by the oral scanning and feedback module (103). The oral data aggregation module (104) simultaneously accepts oral health data which is uploaded by a plurality of user(s) and checks the validity of the uploaded data to ensure that the data is uploaded from authorized devices, in a pre-defined standard format and is in accordance with conventional privacy regulations. The oral health data which is stored in the dynamic oral data aggregation module (104) may be encrypted to ensure additional privacy and security and may be accessed by a pre-defined number of designated authority(ies).

According to one or more embodiments of the present invention, the oral health data stored in the dynamic oral data aggregation module (104) may be retrieved by a disease classifier module (105) which pre-processes the oral health data by extracting specific features from the oral health data such as the rate of change of gas concentrations in a breath sample, consider specific sections of the scan corresponding to different locations of the mouth such as the teeth, tongue, gums or the roof of the mouth. Further, at least one machine learning and/or pattern recognition algorithm(s) is applied to the pre-processed oral health data to detect the presence or absence of at least one disease(s). Conclusively, the output yielded by the disease classifier module (105) comprises an objective classification indicating the presence or absence of one or more disease(s), a level of confidence in the disease classification result and an exact location of one or more disease indicators in the user(s)' mouth.

According to one or more embodiments of the present invention, the oral health data which is retrieved from the oral data aggregation module (104) maybe employed by medical researchers and professionals for performing advanced research and analysis, analyzing disease(s) trends through the statistical data obtained from the oral data aggregation module (104), develop new disease(s) classification and detection models using at least one machine learning and/or pattern recognition algorithm and/or modify the existing disease(s) classification and detection models for improving the performance of the system (100).

According to one or more embodiments of the present invention, the system (100) comprises a personal health advice generation module (106) which analyzes the results obtained from the disease classifier module (105) and provides a personalized primary level advice to the user(s). The personalized advice generated by the personal health advice generation module (106) may suggest specific actions that may be undertaken by the user(s) to prevent the onset or worsening of one or more disease(s) detected at the disease classifier module (105). The personal health advice generation module (106) may also suggest details regarding at least one specialized caretaker(s) depending on one or more disease(s) detected at the disease classifier module (105). The output generated by the personal health advice generation module (106) maybe displayed on the user interface device (102) in the form of text or a plurality of multi-media formats.

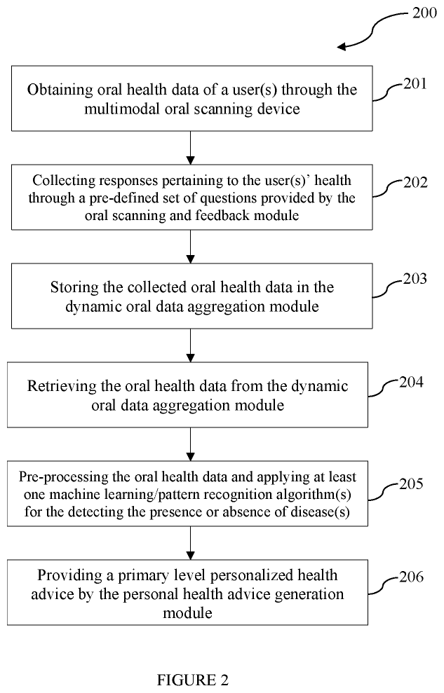

FIG. 2 illustrates a method for providing personal health advice for a plurality of user(s) based on the oral health data which is obtained using the multimodal oral scanning device (101). The method (200) comprises the steps of obtaining oral health data of a user(s) through the multimodal oral scanning device (101) in step (201), wherein the real-time guidance and feedback is provided to the user(s) through the oral scanning and feedback module (103) present in the user interface device (102). In step (202), responses pertaining to the user(s)' health is collected through a pre-defined set of questions provided by the oral scanning and feedback module (103).

The oral health data which is collected from a plurality of user(s) is stored in the dynamic oral data aggregation module (104) in step (203) and is accessed or retrieved from the dynamic oral data aggregation module (104) for further processing in step (204). The retrieved oral health data is pre-processed at the disease classifier module (105) and at least one machine learning and/or pattern recognition algorithm(s) is implemented to detect the presence or absence of one or more disease(s) in step (205). Based on the disease(s) detected by the disease classifier module (105) in step (205), the personal health advice generation module (106) provides a primary level personalized health advice to the user(s) which may be displayed on the user interface device (102) in the form of text or a plurality of multi-media formats.

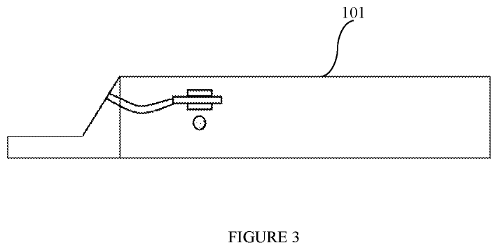

FIG. 3 illustrates a side view of the multimodal oral scanning device which is employed for scanning and collecting oral health data for a plurality of user(s). The multimodal oral scanning device (101) is a handheld, re-chargeable device comprising a plurality of sensors such as camera, breath sensor, temperature sensor, at least one accelerometer(s) and so on. The multimodal oral scanning device (101) is integrated with a user interface device (102) through a wireless network infrastructure which enables a plurality of user(s) to view the status of their oral health data and take necessary actions whenever required.

The present invention provides a system and method which is employed to detect a plurality of health problems based on the oral health data obtained from a plurality of user(s) and provide a personalized primary level advice to the user(s) regarding their overall health. This portable, user-friendly and cost-effective solution enables doctors to monitor their patient(s)' health from a remote location. Further, the system is modularly designed to accommodate the latest technology and infrastructure for optimum usage.

While at least one exemplary embodiment has been presented in the foregoing detailed description, it should be appreciated that a vast number of variations exist.

### REFERENCE Numbers

| | Reference |
|---|---|
| Components | Numbers |
| System | 100 |
| Multimodal oral scanning device | 101 |
| User interface device | 102 |
| Oral scanning and feedback module | 103 |
| Dynamic oral data aggregation module | 104 |
| Disease classifier module | 105 |
| Personal health advice generation module | 106 |

We claim:

1. A system for providing personal health advice based on multimodal oral sensing data, the system (100) comprising:
a. a multimodal oral scanning device (101) for scanning and collecting a plurality of oral health data, wherein the multimodal oral scanning device (101) is a handheld device comprising a plurality of sensors;
b. a user interface device (102) for interfacing with the multimodal oral scanning device (101), wherein the oral health data collected by the multimodal oral scanning device (101) is transmitted to the user interface device (102) through a wireless network infrastructure;
c. an oral scanning and feedback module (103) for providing real-time guidance and feedback to a user(s) regarding the optimum usage of the multimodal oral scanning device (101), wherein the oral scanning and feedback module (103) is interfaced with the user interface device (102);
d. a dynamic oral data aggregation module (104) for receiving and storing the oral health data of a plurality of user(s) from the oral scanning and feedback module (103), wherein the oral health data stored in the dynamic oral data aggregation module (104) is accessed by a pre-defined number of designated authority(ies);
e. a disease classifier module (105) for pre-processing the oral health data present in the dynamic oral data aggregation module (104), wherein the pre-processed oral health data is subjected to at least one machine learning and/or pattern recognition algorithm(s) to detect the presence or absence of at least one disease;
f. a personal health advice generation module (106) for analyzing the results obtained from the disease classifier module (105) and providing primary level advice to the user(s) depending on the results obtained in the disease classifier module (105).

2. The system as claimed in claim 1, wherein the oral scanning and feedback module (103) provides real-time guidance and feedback to the user(s) in a plurality of text and multi-media formats.

3. The system as claimed in claim 1, wherein the scanned oral health data is transmitted form the oral scanning and feedback module (103) to the dynamic oral data aggregation module (104) over a secure cloud-based infrastructure.

4. The system as claimed in claim 1, wherein the dynamic oral data aggregation module (104) accepts oral health data which is uploaded by a plurality of user(s) and processes the data simultaneously.

5. The system as claimed in claim 1, wherein the disease classifier module (105) retrieves oral health data from the oral data aggregation module (104) and pre-processes the data to extract specific features for optimum disease classification.

6. The system as claimed in claim 1, wherein the output yielded by the disease classifier module (105) comprises:
a. an objective classification indicating the presence or absence of one or more diseases;
b. a level of confidence in the disease classification result; and
c. an exact location of one or more disease indicators in the user(s)' mouth.

7. The system as claimed in claim 1, wherein the oral health data which is retrieved from the oral data aggregation module (104) is employed by medical researchers and professionals for:
a. performing advanced research and analysis;
b. analyzing disease(s) trends through the statistical data obtained from the oral data aggregation module (104);
c. develop new disease(s) classification and detection models using at least one machine learning and/or pattern recognition algorithm; and
d. modify the existing disease(s) classification and detection models for improving the performance of the system (100).

8. A method for providing personal health advice based on multimodal oral sensing data, the method (200) comprising the steps of:
a. obtaining oral health data of a user(s) through the multimodal oral scanning device (101), wherein real-time guidance and feedback is provided to the user(s) through the oral scanning and feedback module (103) present in the user interface device (102);
b. collecting responses pertaining to the user(s)' health through a pre-defined set of questions provided by the oral scanning and feedback module (103);
c. storing the collected oral health data in the dynamic oral data aggregation module (104);
d. retrieving the oral health data from the dynamic oral data aggregation module (104);
e. pre-processing the oral health data at the disease classifier module (105) and applying at least one machine learning and/or pattern recognition algorithm(s) for the detecting the presence or absence of one or more disease(s);
f. providing a primary level personalized health advice by the personal health advice generation module (106) based on the inputs received from the disease classifier module (105).
